(19) United States
(12) Patent Application Publication
(10) Pub. No.: US 2026/0066103 A1
(43) Pub. Date: Mar. 5, 2026

(54) COMPUTERIZED SYSTEM AND METHOD FOR FULL PATIENT FLOW MANAGEMENT IN AN EMERGENCY DEPARTMENT

(71) Applicant: SHEBA IMPACT LTD., Ramat Gan (IL)

(72) Inventors: Haggai EDEN, Ramat Gan (IL); Gal BEN-HAIM, Ramat Gan (IL); Yiftach BARASH, Ramat Gan (IL); Yitshak KREISS, Shoham (IL); Vera SORIN, Ness Ziona (IL)

(21) Appl. No.: 19/385,518

(22) Filed: Nov. 11, 2025

Related U.S. Application Data
(63) Continuation of application No. PCT/IL2025/050395, filed on May 11, 2025.
(60) Provisional application No. 63/645,893, filed on May 12, 2024.

(51) Int. Cl. G16H 40/20; G16H 10/20; G16H 10/60; G16H 15/00

(52) U.S. Cl. CPC G16H 40/20; G16H 10/20; G16H 10/60; G16H 15/00

(57) ABSTRACT

Provided herein are computerized system and method for full patient flow management through an emergency department process, integrating multi-agents LLM. The system and method utilize a plurality of LLM-based agents configured to perform one or more of: extract relevant medical information and generate a summarized medical history; interactively questioning the subject regarding its medical history and regarding the main complaint(s); provide recommendations regarding various medical examinations; process the summarized medical history and the results of the recommended medical examination(s) and; provide further summary, recommendations or instructions, regarding diagnosis and a decision regarding discharge or admission of the subject.

### CROSS REFERENCE TO RELATED APPLICATIONS

This application is a Track One Bypass Continuation of PCT Patent Application No. PCT/IL2025/050395 having International filing date of May 11, 2025, which claims the benefit of priority of U.S. Provisional Patent Application No. 63/645,893, filed May 12, 2024, the contents of which are all incorporated herein by reference in their entirety

### TECHNICAL FIELD

The present disclosure relates generally to computerized medical systems. More specifically but not exclusively to computerized system and method for full patient flow management through an emergency department process integrating multi-agents LLM.

### BACKGROUND

An emergency department (ED) in a hospital provides primary care to patients and in many cases an immediate medical care for acute illnesses and injuries requiring urgent attention. The Emergency department is often a patient's first point of contact with the hospital requiring collection of medical history under stressful circumstances. This initial encounter serves as a critical information gathering opportunity where the emergency department staff must quickly establish the patient's medical background and condition to provide appropriate and safe emergency care.

Currently, the emergency department faces significant challenges such as overcrowding which leads to delayed patient care, incomplete and/or disorganized patient data which require the medical professionals and physicians to often spend long time on establishing the medical background of the patient, limited time for accurate triage which may result in suboptimal decisions and the like.

Thus, there is a need in the art for system and method for enhancing patient flow in the emergency department, to provide a medical history, an anamnesis and recommending treatment in a rapid yet reliable manner, thereby minimizing physicians-computer time allowing maximal physician-patient time.

### SUMMARY

According to some embodiments, there are provided herein computerized systems and methods for full patient flow management through an emergency department process, integrating multi-agents LLM. In some embodiments, the systems and methods utilize a plurality of LLM-based agents configured to perform one or more of: extract relevant medical information and generate a summarized medical history of a subject; interact with the subject by interactively questioning the subject regarding its medical history and regarding the main complaint(s) (medical condition for which the subject arrived to the emergency department); provide recommendations regarding various medical examinations (such as laboratory examinations/test and/or imaging examinations/test, etc.); receive the results of said recommended medical examinations; process the summarized medical history and the results of the recommended medical examination(s) and optionally further examination results that may have been conducted; provide further summary, recommendations or instructions, including, for example a diagnosis and a decision regarding admission of the subject to one of a hospital department, optionally with a treatment plan or discharge of the subject, optionally with a treatment plan.

According to some embodiments, advantageously, the system and method enables minimizing the physicians-computer time allowing maximal physician-patient time, thereby enhancing accuracy and efficiency of patient flow management in ER settings.

According to some embodiments, advantageously, the systems and methods disclosed herein increase treatment safety, by reducing misdiagnosis rate, enhancing treatment time (in particular, in un-noticed emergency states), reducing false or un-needed examinations.

In some embodiments, the integration of LLM-based agents in the system and methods disclosed herein advantageously provide a reliable summarized medical history of the subject, in a relatively short time.

In some embodiments, the system and method advantageously allow reducing load and pressure on the medical staff in overcrowding conditions, such as ER environment, by facilitating and conducting the full patient flow ER management process.

According to some embodiments, in one aspect, a computerized system for full patient flow management through an emergency department process is presented herein. The system includes one or more processing units executing code instructions configured to:
receive results of preliminary measurements obtained from a subject in the emergency department;
interactively provide queries to the subject and generate a comprehensive anamnesis utilizing:
a background LLM-based agent, configured to provide queries related to the medical history of the subject and receive medical history related input from the subject; and
an anamnesis LLM-based agent configured to provide queries related to complaint(s) of the subject, and receive complaint related input from the subject;
   wherein the comprehensive anamnesis is generated based on the complaint related input from the subject and the preliminary measurements;
generate potential diagnosis utilizing a diagnosis LLM-based agent, based on the comprehensive anamnesis;
output recommendations regarding one or more medical examinations, utilizing a test recommender LLM-based agent, said agent is configured to receive the generated potential diagnosis and output recommended medical examinations for confirming or ruling-out said potential diagnosis;
receive results of one or more of the recommended medical examinations; and
generate a summary utilizing a decision LLM-based agent based on the recommended medical examinations results and the comprehensive anamnesis, wherein the summary includes:
   a differential diagnosis; and/or
   a recommendation regarding admission or discharge of the subject from the emergency department.

According to some embodiments, the one or more processing units are further configured to execute code instructions configured to:
upon receiving an ID of the subject, extract medical information of said subject from a medical information system; and
generate and store, on a database, a summarized medical history of the subject, by a pre-summary LLM-based agent comprising a plurality of collecting agents, said collecting agents include one or more LLM-based agents and/or one or more extracting agents, each collecting agent is configured to extract and summarize information related to a defined subject-related parameter, said pre-summary LLM-based agent generates the summarized medical history based on the summaries provided by the collecting agents.

According to some embodiments, the one or more processing units are further configured to process the summarized medical history and preliminary measurements results to identify medical information relevant to the preliminary measurements results.

According to some embodiments, the background LLM-based agent is further configured to verify and/or update the information in the processed summarized medical history, based on the medical history related input.

According to some embodiments, the comprehensive anamnesis is further based on the verified and/or updated processed summarized medical history.

According to some embodiments, the potential diagnosis is further based on the verified and/or updated processed summarized medical history.

According to some embodiments, the decision is further based on the verified and/or updated summarized medical history.

According to some embodiments, the defined medical parameters may include such parameters as, but not limited to: chronic conditions, past surgical history, changes and discontinuations of medications, allergies, medical history, acute medications, ambiguous medications, timeline medical history, review of biological systems, imaging and diagnostic results. Each possibility is a separate embodiment.

According to some embodiments, the summarized medical history is generated in about 5-10 minutes.

According to some embodiments, the summary further includes:
instructions to repeat execution of the anamnesis LLM-based agent, the diagnosis LLM-based agent, and/or the test recommender LLM-based agent; and/or
instructions to add or repeat recommended medical examinations.

According to some embodiments, the one or more processing units are further configured to execute code instructions configured to receive results of a physical examination, after generating the comprehensive anamnesis.

According to some embodiments, the physical examination is done by a human clinician or by an IOT medical device.

According to some embodiments, the recommended medical examinations may include lab tests, imaging, and/or physical examinations.

According to some embodiments, the one or more processing units further executes code instructions configured to analyze the imaging examinations.

According to some embodiments, the one or more processing units execute code instructions configured to present the recommended medical examinations and summary via an interface module, to the subject and/or to a health care provider.

According to some embodiments, the preliminary measurements may include body temperature, heart rate, oxygen saturation, diastolic blood pressure, systolic blood pressure, sugar level, pain level and weight.

According to some embodiments, the preliminary measurements may be conducted automatically or semi-automatically by an IOT medical device or by a health care provider.

According to some embodiments, the one or more processing units execute code instructions configured to recommend a treatment plan during the subject's stay at the emergency department, or after the one or more medical examinations results are received, utilizing a drug plan LLM-based agent.

According to some embodiments, the treatment plan includes a drug treatment.

According to some embodiments, the decision LLM-based agent is configured to receive an approval or change from a human clinician to the recommendation regarding the admission or discharge of the subject.

According to some embodiments, the anamnesis LLM-based agent is configured to receive a human clinician approval or changes regarding the complaint related input from the subject and/or amending the provided anamnesis.

According to some embodiments, the test recommender LLM-based agent is configured to receive approval or changes or additions from a human clinician regarding the one or more medical examinations recommended.

According to some embodiments, the one or more processing units execute code instructions configured to review one or more of the LLM-based agents operation and output and provide a score to the one or more LLM-based agents for said operation and output, utilizing an evaluation LLM-based agent.

According to some embodiments, the evaluation LLM-based agent is configured to evaluate the output of one or more LLM-based agents with respect to content structure and linguistic integration using guardrails implemented to ensure that the LLM-based agent operates safely, ethically and in accordance with the LLM-based agent's defined objectives.

According to some embodiments, the LLM in the computerized system is Generative Pretrained Transformer (GPT).

According to some embodiments, in a second aspect a computerized method for full patient flow management through an emergency department process is presented herein. The method includes:
receiving results of preliminary measurements obtained from the subject in the emergency department;
interactively providing queries to the subject and generating a comprehensive anamnesis utilizing:
   a background LLM-based agent, configured to provide queries related to the medical history of the subject and receive medical history related input from the subject; and
   an anamnesis LLM-based agent configured to provide queries related to complaint(s) of the subject, and receive complaint related input from the subject;
   wherein the comprehensive anamnesis is generated based on the complaint related input from the subject and the preliminary measurements;
generating potential diagnosis utilizing a diagnosis LLM-based agent, based on the comprehensive anamnesis;
outputting recommendations regarding one or more medical examinations, utilizing a test recommender LLM-based agent, said agent is configured to receive the generated potential diagnosis and output recommended medical examinations for confirming or ruling-out said potential diagnosis;
receiving results of one or more of the recommended medical examinations; and
generating a summary utilizing a decision LLM-based agent based on the recommended medical examinations results and the comprehensive anamnesis, wherein the summary may include:
   a differential diagnosis; and/or
   a recommendation regarding admission or discharge of the subject from the emergency department.

According to some embodiments, the method may further includes:
upon receiving an ID of a subject, extracting medical information of said subject from a medical information system;
generating and storing, on a database, a summarized medical history of the subject, by a pre-summary LLM-based agent comprising a plurality of collecting agents, said collecting agents may include one or more LLM-based agents and/or one or more extracting agents, each collecting agent is configured to extract and summarize information related to a defined subject-related parameter, said pre-summary LLM-based agent generates the summarized medical history based on the summaries provided by the collecting agents.

According to some embodiments, the method may further include processing the summarized medical history and preliminary measurements results by the pre-summary LLM-based agent to identify medical information relevant to the preliminary measurements results.

According to some embodiments, the background LLM-based agent is further configured to verify and/or update the information in the processed summarized medical history, based on the medical history related input.

According to some embodiments, the comprehensive anamnesis is further based on the verified and/or updated processed summarized medical history.

According to some embodiments, the potential diagnosis is further based on the verified and/or updated processed summarized medical history.

According to some embodiments, the summary is further based on the verified and/or updated summarized medical history.

According to some embodiments, the summary may further include:
instructions to repeat execution of the background LLM-based agent, the anamnesis LLM-based agent, the diagnosis LLM-based agent, and/or the test recommender LLM-based agent; and/or
instructions to add or repeat recommended medical examinations.

According to some embodiments, the summary is generated after receiving results of the one or more medical examinations or the summary is regenerated after receiving the results of the added or repeated recommended medical examinations.

According to some embodiments, the method further comprising receiving results of a physical examination, after generating the comprehensive anamnesis.

According to some embodiments, the physical examination is done by a human clinician or by an IOT medical device.

According to some embodiments, the method may further include receiving an approval or change from a human clinician to the recommendation regarding the admission or discharge of the subject.

According to some embodiments, the method may further include recommending a treatment plan.

According to some embodiments, the treatment plan may include a drug treatment.

According to some embodiments, the treatment plan may be recommended during the subject's stay at the emergency department, or after receiving the one or more medical examination results.

According to some embodiments, the one or more medical examinations comprising lab tests imaging and/or physical examinations.

According to some embodiments, the method may further include determining urgency based on the subject's complaint, anamnesis and identified medical information relevant to the preliminary measurements results, after generating the comprehensive anamnesis.

According to some embodiments, the method may further include reviewing one or more of the LLM-based agents operation and output and providing a score to the one or more LLM-based agents for said operation and output, utilizing an evaluation LLM-based agent.

According to some embodiments, reviewing one or more LLM-based agents operation and output may include evaluating the output of the respective LLM-based agent with respect to content structure and linguistic integration using guardrails implemented to ensure that the LLM-based agent operates safely, ethically and in accordance with the LLM-based agent's defined objectives.

According to some embodiments, the method may further include receiving further complaint input from the subject by a human clinician and/or receiving an amended anamnesis by said clinician.

According to some embodiments, the method may further include receiving approval or changes or additions from a human clinician regarding the one or more medical examinations recommended.

According to some embodiments, in a third aspect a non-transitory computer-readable medium full patient flow management through an emergency department process is presented herein. The non-transitory computer-readable medium having stored thereon instructions that cause a processor to:
receive results of preliminary measurements obtained from a subject in the emergency department;
interactively provide queries to the subject and generate a comprehensive anamnesis utilizing:
a background LLM-based agent, configured to provide queries related to the medical history of the subject and receive medical history related input from the subject; and
an anamnesis LLM-based agent configured to provide queries related to complaint(s) of the subject, and receive complaint related input from the subject;
   wherein the comprehensive anamnesis is generated based on the complaint related input from the subject and the preliminary measurements;
generate potential diagnosis utilizing a diagnosis LLM-based agent, based on the comprehensive anamnesis;
output recommendations regarding one or more medical examinations, utilizing a test recommender LLM-based agent, said agent is configured to receive the generated potential diagnosis and output recommended medical examinations for confirming or ruling-out said potential diagnosis;
receive results of one or more of the recommended medical examinations; and
generate a summary utilizing a decision LLM-based agent based on the recommended medical examinations results and the comprehensive anamnesis, wherein the summary may include:
   a differential diagnosis; and/or
   a recommendation regarding admission or discharge of the subject from the emergency department.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1A schematically shows a diagram of a computerized system for full patient flow management through an emergency department utilizing multi-agents LLM, according to some embodiments;

FIG. 1B schematically shows a diagram of an architecture of multi-agents LLM integrated in a computerized system for full patient flow management through an emergency department process, according to some embodiments;

FIG. 1C schematically shows a diagram of an architecture flow including inputs and outputs of each agent, in a computerized system for full patient flow management through an emergency department process, according to some embodiments;

FIG. 2 schematically shows a flowchart of computerized system for full patient flow management through an emergency department process integrating multi-agents LLM, according to some embodiments;

FIG. 3 schematically shows a flowchart of an example of a subject received at an emergency department and treated by a computerized system for full patient flow management through an emergency department process integrating multi-agents LLM, from the subject interface device end, according to some embodiments;

FIG. 4 schematically shows a clinician interface device screen with prioritization of subjects, according to some embodiments;

FIG. 5 schematically shows a comparison between results a of medical examination recommendations made by LLM and physicians;

FIG. 6A schematically shows the rate of matching between the recommended differential diagnosis of the LLM and the physician diagnosis;

FIG. 6B schematically shows the rate of matching priority differential diagnoses between the LLM and physicians;

FIG. 7A schematically shows the rate of agreement on patient discharge decisions between the LLM and physicians; and

FIG. 7B schematically shows the discrepancies in patient discharge decisions where the LLM and physicians disagreed (n=20).

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

As used herein, the term “about” may be used to specify a value of a quantity or parameter (e.g. the length of an element) to within a continuous range of values in the vicinity of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

As used herein, according to some embodiments, the term “prompt” related to input text or instructions given to an LLM model to generate a response.

As used herein, according to some embodiments, the term “prompt engineering” relates to the practice of designing, refining and optimizing input to LLM/AI models to generate desired outputs.

As used herein, according to some embodiments, the term “agent” relates to an autonomous or semi-autonomous software system designed to perceive its environment, make decisions based on program objectives and take actions such as interact with external tools, systems and/or environments, create execution plans, and the like, to achieve specific goals.

As used herein, according to some embodiments, the term “LLM-based agent” relates to an autonomous or semi-autonomous software system using the LLM as their reasoning engine, and which are able to take actions such as interact with external tools, systems and/or environments, create execution plans and the like. LLM-based agents integrates the abilities of agents with cognitive and linguistic abilities of LLMs creating agents with high flexibility and understanding capabilities.

As used herein, the term “emergency department”, “ED”, “emergency room” and “ER” may be used interchangeably.

According to some embodiments, provided herein are computerized systems and methods for full patient flow management through an emergency department process, wherein the systems and methods integrates multi-agents LLM.

According to some embodiments, the computerized system and method are configured to: summarize medical history, conduct anamnesis, triage patients by identifying urgency, and/or recommend necessary examinations (such as blood tests and imaging). After every stage, the system is configured to evaluate the situation of the patient (also referred to herein as “subject”), according to new and/or additional information received/obtained, in order to provide more accurate diagnosis and recommendations. After the results of the examinations are received a summary, including one or more recommendations, is generated. Such summary may include, for example, but not limited to: recommendation to repeat one or more of the previous stages, recommendation to repeat or add new recommended examinations, provide a diagnosis, recommendation to discharge or hospitalize the patient, optionally together with a treatment plan, or any combinations thereof. Each possibility is a separate embodiment.

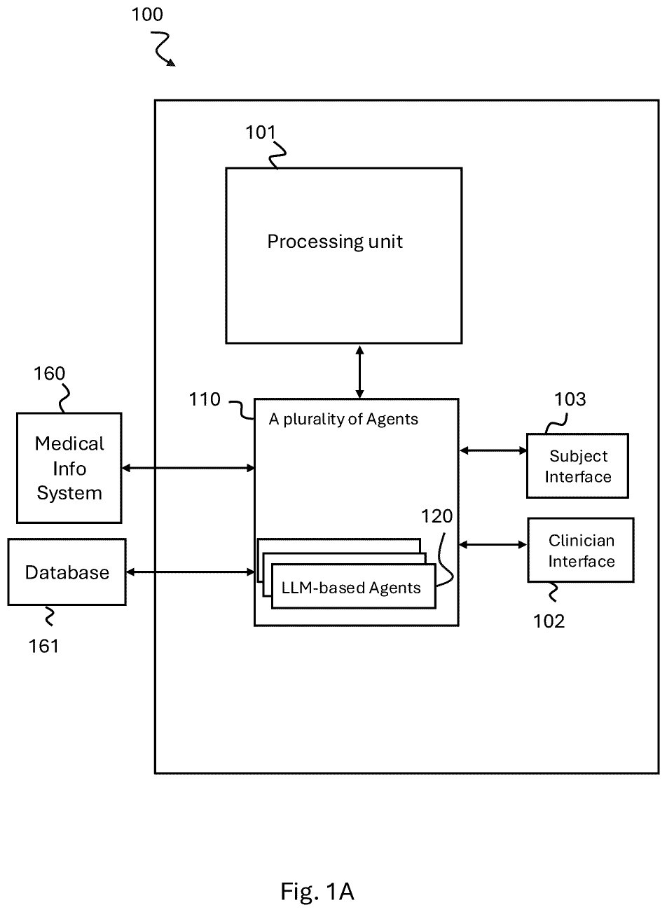

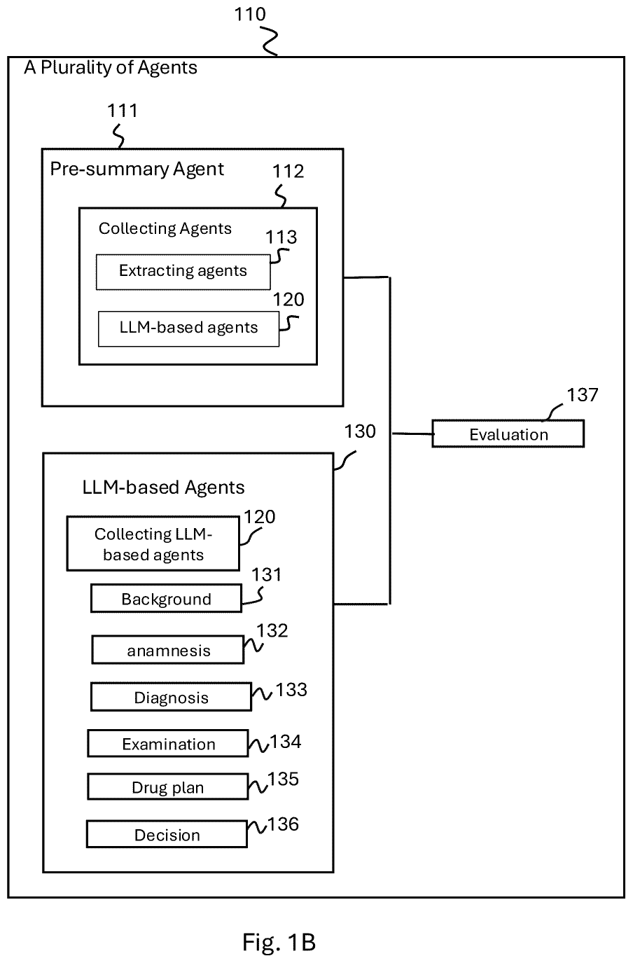

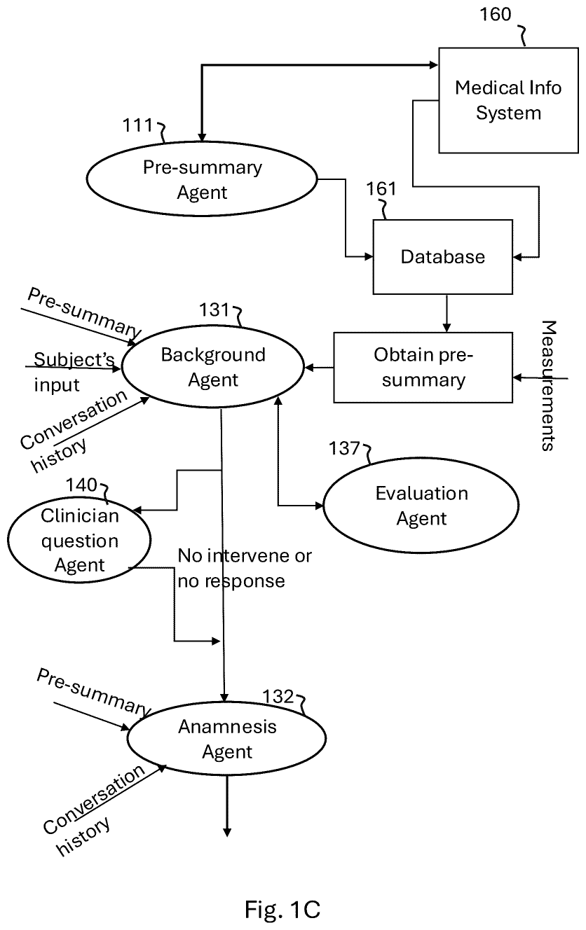

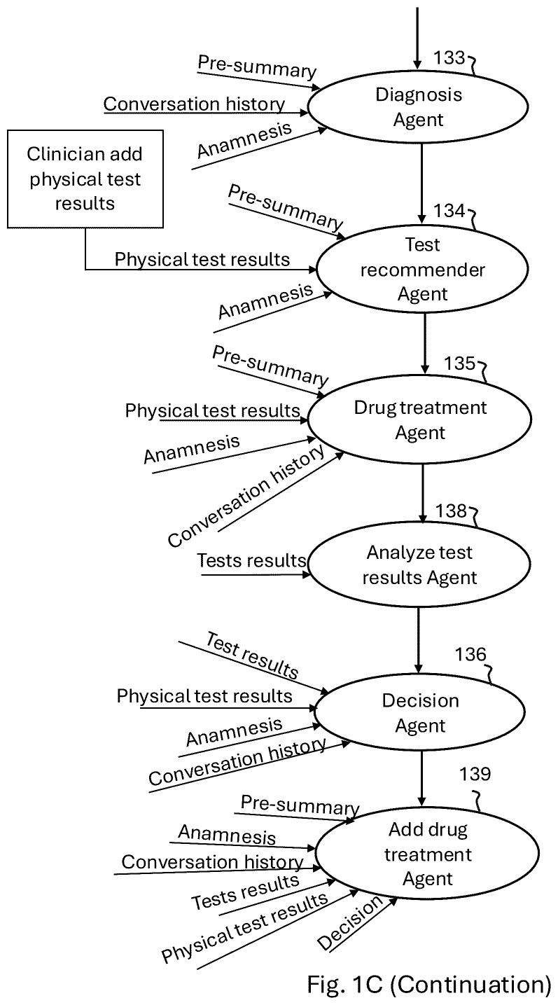

Reference is now made to FIG. 1A which schematically shows a diagram of a computerized system for full patient flow management through an emergency department process integrating multi-agent large language model (LLM), according to some embodiments. As shown in FIG. 1A, system 100 includes a processing unit 101 (which may include one or more functionally associated processors), which are configured to execute a plurality of agents 110, including LLM-based agents 120, as further detailed herein below. In some embodiments, system 100 may further include one or more clinician/health care provider interface device 102 and one or more subject interface device 103. According to some embodiments, the health care provider and/or the subject interface device includes a display, a monitor, a screen, a user interface input device, such as keyboard and mouse, a touch screen, and the like, or any combinations thereof. In addition, the input device may further include a speaker and a microphone for receiving audio input from the subject and outputting audio output, as a speech instructions or conversation from system 100. In some embodiments the health care provider and/or the subject interface device includes a mobile device (for example a smartphone, a tablet and the like). In some embodiments, system 100 may include mobile connectivity capabilities such that the subject may use any mobile device as an interface device. In some embodiments, Processing unit 101 and the plurality of agents 110 are configured to access and interact with a medical information system 160 and a database 161. The medical information system and the database may be stored on a local or remote server and may be functionally or communicatively associated with the system.

Reference is now made to FIG. 1B which schematically shows a diagram of an architecture of a plurality of agents, including LLM-based agents integrated, in a computerized system for full patient flow management through an emergency department process, and to FIG. 1C which schematically shows a diagram of an architecture flow including inputs and outputs of each agent, according to some embodiments. Plurality of agents 110 include, for example, a pre-summary agent 111 which can incorporate or include one or more collecting agents 112, which are configured to collect subject-related information. The collecting agents 112 may include one or more extracting agents 113, which may not necessarily be LLM-based, and/or one or more LLM-based agents 120. Each of the collecting agents may extract data regarding a defined subject-related parameter. For example, LLM-based agents 120 the collecting agents may include LLM-based agents configured to extract data regarding one or more subject parameters of the subject, including for example, but not limited to: chronic conditions, past surgical history, changes and discontinuations of medicaments or treatment plans, allergies, medical history, acute medications, ambiguous medications, timeline medical history, review of biological systems, previous imaging and/or diagnostic results, and the like. Each of the collecting LLM-based agents may be prompt engineered to receive as an input the data extracted by other agents and provide a summary regarding the specified subject-related parameter defined for said collecting LLM-based agent. The Pre-summary agent may be prompt engineered to receive as input the summaries generated/provided by the collecting agents and generate a summarized medical history of the subject based thereon.

According to some embodiments, plurality of agents 110 may further include one or more of the following agents: a background LLM-based agent 131, configured to verify and update the summarized medical history generated by pre-summary agent 111. Background LLM-based agent 131 may be prompt engineered to receive the summarized medical history, initial measurements obtained from the subject (during acceptance to the ER), and information received from the subject during interactive conversation with a virtual avatar representing the system. Background LLM-based agent 131 may be prompt engineered to process the summarized medical history according to the measurements obtained from the subject and to verify and/or update the preprocessed summarized medical history. In some embodiments, when there is no summarized medical history, for example in case of a subject with no records in the medical information system 160, the Background LLM-based agent 131 generates a summarized medical history according to the measurements obtained from the subject and input received from the subject regarding the subject's medical history and lifestyle during the interactive conversation with the subject.

According to some embodiments, a clinician question agent 140, which is an optional agent, may be configured to receive a summary of the verified and/or updated summarized medical history with the conversation history as input and may present to a clinician via clinician interface device 102 the summary of the verified and/or updated summarized medical history with the conversation history, for amendments. The clinician may intervene and rephrase questions or add additional questions for asking the subject.

According to some embodiments, Anamnesis LLM-based agent 132 is configured for generating a comprehensive anamnesis of the subject. To this aim, in some embodiments, Anamnesis LLM-based agent 132 may be prompt engineered to receive the verified/updated processed summarized medical history and conversation history with the subject and to question the subject regarding the main complaint for which he came to the ED. In some embodiments, the Anamnesis LLM-based agent 132 may interactively provide queries to the subject regarding the complaint(s) for which he arrived to the ER. In some embodiments the Anamnesis LLM-based agent may be configured to receive vocal input from the subject in response to the questions and to perform voice analysis to said vocal input to further analyze the subject condition. In some embodiments, the Anamnesis LLM-based agent may further be configured to receive visual input of the subject such as an image of the subject captured during the questioning regarding the medical history or main complaint of the subject and to perform image analysis which may indicate a medical situation of the subject (for example, paleness, redness, sweating and the like). The voice and image analysis results may affect the urgency determination later determined by the system. Thus, the Anamnesis LLM-based agent 132 is configured to generate a comprehensive anamnesis based on the input and questioning of the subject.

According to some embodiments, Diagnosis LLM-based agent 133 is configured to generate a possible/potential/optional/initial diagnosis. Diagnosis LLM-based agent 133 may be prompt engineered to receive the comprehensive anamnesis (together with or instead of the conversation history with the subject) and the validated/updated processed summarized medical history, and based thereon, output a potential diagnosis. According to some embodiments, after the generation of the anamnesis, a clinician may intervene and conduct a physical examination and feed the results of the physical examination to system 100.

According to some embodiments, Test recommender LLM-based agent 134 is configured to output recommendations for one or more medical examinations (including, tests (such as lab tests (for example, blood tests) and/or examinations (such as, imaging (e.g., MRI, CT, X-Ray), physical examination, medical examinations (e.g., ECG, EEG, etc.), and the like), for confirming or ruling-out the potential diagnosis. In some embodiments, Test recommender LLM-based agent 134 may be prompt engineered to receive as an input the potential diagnosis, comprehensive anamnesis, (together with or instead of the conversation history with the subject) the verified/updated processed summarized medical history, and optionally the results of a physical examination and output the recommended medical tests and examinations. According to some embodiments, the test recommender LLM-based agent may be divided into two or more agents, for example, a laboratory test recommender agent, imaging test recommender agent and the like.

According to some embodiments, optional Drug plan LLM-based agent 135 is configured to generate a drug plan or a treatment plan for the subject. Drug plan LLM-based agent 135 may be prompt engineered to receive as an input the comprehensive anamnesis (together with or instead of the conversation history with the subject) the verified/updated processed summarized medical history, and optionally the results of the recommended medical examinations and output a drug or treatment plan for the subject. In some embodiments, drug plan agent may be a separate agent or may be part of other agents. In some embodiments, when a treatment/drug plan may be necessary immediately and/or before reception of the recommended medical examinations results, the drug/treatment plan may be outputted without receiving the recommended medical examinations results as an input. In this case, once the recommended examination results are available the treatment plan agent 135 may further receive the results as an input and output an updated treatment plan accordingly.

According to some embodiments, an optional agent, Analyze test results LLM-based agent 138 is configured to receive as an input the results of the recommended medical examinations and output and analysis of the results.

According to some embodiments, Decision LLM-based agent 136 is configured to generate a summary, based on the output from one or more of the other agents (e.g., agents 131, 132, 133, 134 and/or 135, and optionally 138). In some embodiments, the summary may include differential diagnosis, a decision regarding admission or discharge of the subject (optionally with a drug/treatment plan), a recommendation to re-execute/repeat operation of one or more of the other agents, a recommendation regarding repeating and/or adding recommended medical examinations, and the like. or any combinations thereof. In some embodiments, Decision LLM-based agent 136 may be prompt engineered to receive as an input the comprehensive anamnesis, (together with or instead of the conversation history with the subject), recommended medical examination results, drug/treatment plan (optionally approved by a human clinician) and optionally the verified/updated processed summarized medical history, and output the summary. In some embodiments, the summary may be presented to the subject. In some embodiments, a treatment plan may be generated or regenerated after the generation of the summary by the drug plan LLM-based agent 135 or by an add drug treatment LLM-based agent 139 configured to receive as an input the verified/updated processed summarized medical history, the comprehensive anamnesis, (together with or instead of the conversation history with the subject), recommended medical examination results (including optionally result of a physical examination) and the summary (including decision of admission or discharge and a differential diagnosis), and output a treatment plan.

In some embodiments, the plurality of agents may further optionally include an evaluation agent 137 which is configured to evaluate the performance of one or more of the other agents. The evaluation agent may an LLM based agent.

According to some embodiments, the suggested output or the performance of one or more of the agents may be supervised by a human expert (for example, a clinician). According to some embodiments, the suggested output of one or more of the agents may be approved by a human expert (for example, a clinician), prior to being executed/used/processed by other agent(s) or prior to being presented.

According to some embodiments, the LLM-based agents 130 may be prompt engineered to receive input from the subject and/or other agents, and each of said agents is configured to output a specified output as described above.

According to some embodiments, processing unit 101 may execute code instructions configured to, upon receiving an ID of a subject, extract medical information of the subject from medical information system 160. According to some embodiments, processing unit 101 may execute code instructions configured to generate a summarized medical history of the subject by pre-summary LLM-based agent 111, including/using a plurality of collecting agents 112 which includes one or more extracting agents 113 and one or more LLM-based agents 120, each collecting agent is configured to extract and summarize information related to a defined subject-related parameter. The pre-summary LLM-based agent 111 generates the summarized medical history, based on the individual summaries provided by all the collecting agents 112. According to some embodiments, each collecting agent 112 is configured to extract and summarize information related to one defined (individual) subject-related parameter.

According to some embodiments, pre-summary LLM-based agent 111 is configured to generate from some or all of the individual summaries, the summarized medical history of the subject, according to a defined scheme.

According to some embodiments, processing unit 101 may execute code instructions configured to store the summarized medical history in database 161. Database 161 may be a local database or a remote database, a cloud database or any type of database.

According to some embodiments, processing unit 101 may execute code instructions configured to receive results of preliminary measurements obtained from the subject in the emergency department. Such measurements may include, for example, but not limited to: body temperature, pulse, oxygen saturation (SpO2), blood pressure, sugar level, weight, and the like. Such measurements may be obtained upon admission to the ED, by a health care provider.

According to some embodiments, processing unit 101 may execute code instructions configured to process the summarized medical history and preliminary measurements results by background LLM-based agent 131, which is configured to identify medical information relevant to the preliminary measurements results.

According to some embodiments, processing unit 101 may execute code instructions configured to interactively question the subject via a subject interface device 103. According to some embodiments, the questioning may be facilitated by two LLM-based agents: a background LLM-based agent 131 which is configured to (interactively) question the subject regarding the subject's history to verify and/or update the information in the processed summarized medical history; and an anamnesis LLM-based agent 132 which is configured to (interactively) question the subject regarding the subject's complaint(s) which led the subject to arrive to the ED. Based upon input from the background LLM-based agent and the anamnesis LLM-based agent a comprehensive anamnesis is generated. According to some exemplary embodiments, questioning regarding the subject's background may first include an introduction of the computerized system to the subject, via a virtual avatar, which may be presented in the subject interface device 103. Such presentation may include, for example, explaining it is a virtual avatar providing healthcare support to the subject, questions regarding the summarized medical history to verify and/or update the information in the summarized medical history and questions relating to the subject lifestyle which may be relevant to medical aspect. For example, such questions mya include, for example, but not limited to questions regarding taking drugs, drinking alcohol, smoking, physical activity training, and diet regimen, and the like. According to some embodiments, in the questioning regarding the subject's background, background LLM-based agent 131 may be configured to decide how many questions to ask the subject based on the subject responses. For example, if a question regarding recent hospitalizations is presented to the subject, and the subject says he was hospitalized recently, background LLM-based agent, is configured to continue asking the subject regarding the recent hospitalizations until the LLM-based agent decides it has enough information regarding the recent hospitalizations. According to some embodiments, background LLM-based agent 131 may be further configured to ask the subject regarding pain, including asking the subject to quantify the level of pain the subject feels at the same moment, for example from 1-10, and asking if the subject is interested in a painkiller to calm the pain. In case the subject is interested in a painkiller(S), background LLM-based agent 131 may be configured to indicate to a clinician that the subject is interested in a painkiller via clinician-interface device 102. The fact that the subject is interested in a painkiller may indicate an acute medical situation which may require further medical intervention. Background LLM-based agent 131 is configured to offer a specific painkiller to the subject such as paracetamol, however, this may be authorized by a clinician. In this case the background LLM-based agent is configured to ask the clinician if the specific painkiller suggested by background LLM-based agent 131 in response to the subject's request is authorized.

According to some embodiments, anamnesis LLM-based agent 132 is configured to receive from background LLM-based agent 131 the validated and/or updated summarized medical history and some or all additional information gathered from the questioning of the subject. According to some embodiments, anamnesis LLM-based agent 132 is configured to question the subject regarding the subject's complaint(s) and the reasons for which the subject arrived at the emergency department. According to some embodiments, anamnesis LLM-based agent 132 is configured to generate a comprehensive anamnesis according to subject's complaint and the verified and/or updated preprocessed summarized medical history.

According to some embodiments, at this stage, a physical examination or other medical examinations may be conducted by a medical device or a human clinician and processing unit 101 may execute code instructions configured to receive the results of such a physical examination from the human clinician via clinician-interface device 102. According to some embodiments, processing unit 101 may execute code instructions configured to generate one or more possible diagnoses by diagnosis LLM-based agent 133, based on the comprehensive anamnesis and validated and/or updated processed summarized medical history and optionally the results of the physical/medical examination. According to some embodiments, processing unit 101 may execute code instructions configured to output recommendations regarding additional examinations by a test recommender LLM-based agent 134 configured to receive the generated possible diagnosis from diagnosis LLM-based agent 133 and provide medical tests and examinations for confirming or ruling-out said possible diagnosis. The medical tests and examinations may include laboratory tests/examinations and imaging examinations such as biochemistry tests, microbiology tests, blood test, swab test, ECG, EEG, EMG, Magnetic Resonance Imaging (MRI), Ultrasound (US), Computerized Tomography (CT), Position Emission Tomography CT (PET-CT). X-ray and the like, or any combinations thereof. According to some embodiments, test recommender LLM-based agent 134 may be further configured to issue/provide a referral for each recommended examination and send the referral to the corresponding unit/department in the hospital responsible for conducting the examination. In addition, according to some embodiments, processing unit 101 may execute code instructions configured to decode the examination by test recommender LLM-based agent 134 or by one of the plurality of agents 110.

According to some embodiments, processing unit 101 may execute code instructions configured to receive the test and examinations results and provide a differential diagnosis by a decision LLM-based agent 136. According to some embodiments, decision LLM-based agent, is configured to or prompt engineered to receive/extract the test and examination results and decoding (optionally decoded by the decision LLM-based agent or by a human clinician) thereof; the comprehensive anamnesis, the verified/updated summarized medical history and optionally the conversation with the subject, and based on the input, provide a differential diagnosis. According to some embodiments, decision LLM-based agent 136, may be configured to present to a clinician via clinician interface device 102 the differential diagnosis for approval and/or additions and/or changes. If it is approved, the decision LLM-based agent may be configured to provide a decision regarding admission of the subject (optionally with a treatment plan) or discharge of the subject (optionally with a treatment plan). According to some embodiments, drug plan LLM-based agent 135 may be configured to provide a treatment/drug plan or one or more specific drugs to the subject before providing a decision regarding admission or discharge of the subject, depending on the subject's condition. According to some embodiments, the drug plan may be presented to a human clinician for approval/changes, via clinician interface device 102.

According to some embodiments, in any stage, a human clinician may supervise or intervene. According to some embodiments, every decision or recommendation of any one of the LLM-based agents is presented first to a clinician for approval and/or additions and/or changes.

According to some embodiments, only after the recommendation and/or decision of the LLM-based agent is approved by a clinician, the decision or recommendation is presented to the subject via subject interface device 103.

According to some embodiments, processing unit 101 may execute code instructions configured to evaluate the performance of each of the agents including the LLM-based agents by evaluation LLM-based agent 137. According to some embodiments, evaluation LLM-based agent 137, is configured to evaluate each agent output and specifically each LLM-based agent output with respect to the content structure and linguistic integration, using guardrails implemented to ensure that the agent operates safely, ethically and in accordance with its defined objectives.

According to some embodiments, the evaluation and guardrails ensure the output of the agents are of high quality and relevancy, accurate and include safety limitations, preventing dangerous or harmful behaviours from the agent. Additionally, monitoring the agent(s) actions and intervening when necessary and limiting the range of actions the agent is authorized to perform enhance safety and accuracy. According to some embodiments, the guardrails are configured to ensure in runtime the agents output is within boundary definitions and prevents wrong content output.

According to some embodiments the evaluation of the system performance and/or of any of the agents may be facilitated by comparing the agent output to one or more gold standard examples.

According to some embodiments, evaluation LLM-based agent 137, may receive an expert's feedback regarding the output of each agent, on-line or offline.

According to some embodiments, the data in the output of one or more of, or of each of the LLM-based agents may be organized at a Fast Healthcare Interoperability Resources (FHIR) structure which is a global standard for holding patients data. According to some embodiments, the FHIR structure may interface with other systems regarding subjects treated in different institutions.

According to some embodiments, processing unit 101 may execute code instructions configured to present to the clinician(s) and or care giver provider via the clinician interface device a sorting of the subjects being treated by the system indicating the stage each of the subjects is and urgency determination of each subject. According to some embodiments, the clinician interface device may manage and prioritize tasks at real time by presenting the subject sorting according to their urgency to the clinician. According to some embodiments, the virtual avatar representing the system may be configured to request an immediate intervention of one or more clinician for example in case of an acute medical situation. The request may be manage by any one of the LLM-based agents recognizing the need for immediate intervention of a clinician, for example, the Background LLM-based agent, the Anamnesis LLM-based agent the test recommender LLM-based agent etc.

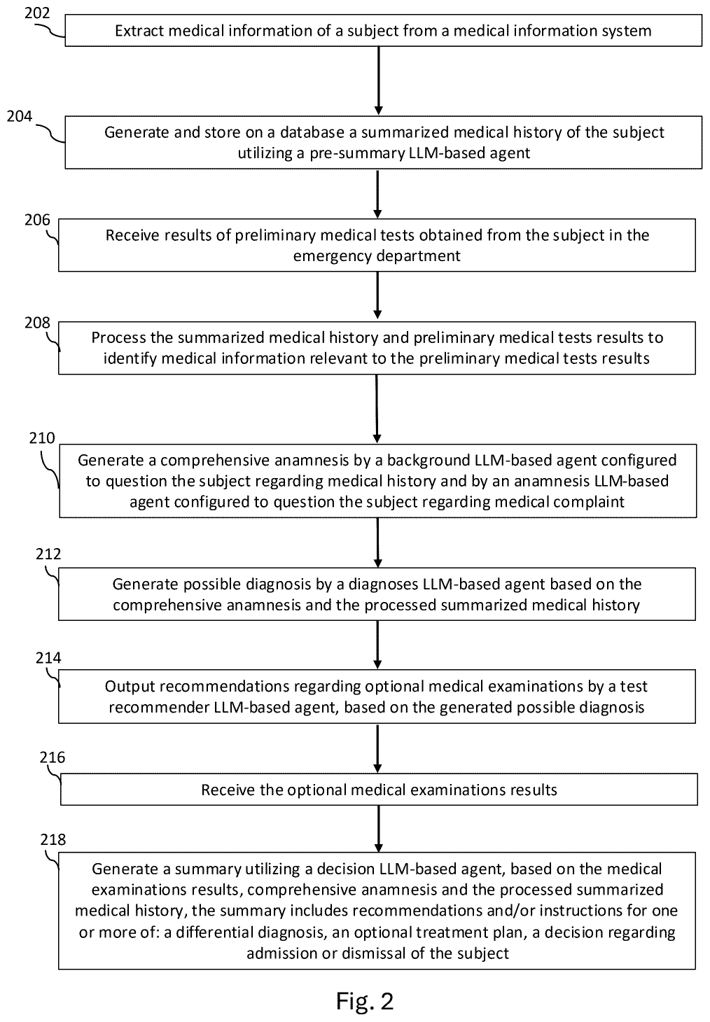

Reference is now made to FIG. 2, which schematically shows a flowchart of an AI based computerized method for full patient flow management through an emergency department process, according to some embodiments. At step 202, upon receiving an ID of a subject, medical information of said subject is extracted from a medical information system (such as medical information system 160) and stored at a database (such as database 161). The database may be a local database or a remote database, including on the cloud. According to some embodiments, the medical information system is the hospital/medical center to which the emergency departments belongs. In addition, the information system may be associated with or a part of a larger medical information system such as of a national healthcare organization.

At step 204, a summarized medical history of the subject is generated and stored by pre-summary LLM-based agent 111 which uses plurality of collecting agents 112 comprising one or more LLM-based agents and/or one or more extracting agents, each of the collecting agent 112 is configured to extract and summarize information related to a defined subject-related parameter. The plurality of collecting agents 112 are configured to work in parallel. According to some embodiments, each collecting agent 112 is configured to extract and summarize information related to a defined subject-related parameter. According to some embodiments, the summarized medical history is generated by pre-summary LLM-based agent 111 based on the summaries provided by collecting agents 112.

According to some embodiments, the medical parameters may include chronic conditions, past surgical history, changes and discontinuations of medications, allergies, medical history, acute medications, ambiguous medications, timeline medical history, review of biological systems, imaging and diagnostic results, and the like. Some or all of the LLM-based agent in the group of agents may be prompt engineered to collect data of one medical parameter and provide a summary of said medical parameter of the subject. According to some embodiments, each summary of a medical parameter may include the name of the medical parameter and the details regarding said medical parameter. For example, the summary of the allergies can include the names of the allergens and the reactions of each of the allergens, the summary of the chronic condition includes the condition details, and the like, or any combinations thereof.

According to some embodiments, the summary of each defined medical parameter may be generated in a JSON format. According to some embodiments, after all the summaries of the different medical parameters are provided, a summarized medical history may be generated by the pre-summary LLM-based agent, wherein the information of the summarized medical history is organized in a defined scheme. According to some embodiments, the summarized medical history may be generated in a JSON format. According to some embodiments, the data in the output of the summaries of each of (or one or more of) the collecting LLM-based agents may be organized at a Fast Healthcare Interoperability Resources (FHIR) structure which is a global standard for holding patients data.

According to some embodiments, advantageously, the use of a plurality of LLM-based or other agents to automatically summarize each medical parameter and to automatically summarize the provided summaries of the medical parameters into one summarized medical history document organized in a defined scheme may advantageously take a few minutes for example, about 3-5 minutes, 6-15 minutes, 8-12 minutes. Each is a separate embodiments.

According to some embodiments, at step 206, results of preliminary measurements obtained from the subject in the emergency department are received. According to some embodiments, the preliminary measurements may include body heat, pulse, saturation, blood pressure, sugar level and weight. According to some embodiments, some or all of the preliminary measurements may be conducted in a one click operation by an IOT medical device.

According to some embodiments, at step 208, the summarized medical history and preliminary measurements results are processed by an agent, which may be the pre-summary/background LLM-based agent 111/131 to identify medical information which may be relevant to the preliminary measurements results.

According to some embodiments, at step 210, the subject is interactively questioned, for example, via a subject-interface device by background LLM-based agent 131, configured to question regarding the subject's history in order to verify and/or update the information in the processed summarized medical history. Additionally, an anamnesis LLM-based agent 132 questioning regarding the subject's complaint. A comprehensive anamnesis is generated according to subject's complaint and the verified and/or updated preprocessed summarized medical history, by anamnesis LLM-based agent 132.

According to some embodiments, at step 212, possible/potential/initial diagnosis is generated by diagnosis LLM-based agent 133, based on the comprehensive anamnesis and the verified and/or updated preprocessed summarized medical history. According to some embodiments, the diagnosis LLM-based agent is configured to specifically explain each diagnosis generated and/or highlight what in the medical history, preliminary measurements and/or comprehensive anamnesis led to the respective diagnosis.

According to some embodiments, at step 214, recommendations regarding medical tests and examinations are outputted by a test recommender LLM-based agent 134, which may be prompt engineered to receive the generated possible diagnosis and output examinations for confirming or ruling-out said possible diagnosis.

According to some embodiments, at step 216, the examinations results are received (for example, automatically). According to some embodiments, at step 218, a summary is generated and presented to the subject via the subject-interface device 103 by decision LLM-based agent 136, based on the examinations results, comprehensive anamnesis and the verified and/or updated processed summarized medical history. The summary may include a differential diagnosis, a decision regarding admission of the subject, optionally, with a treatment plan, or discharge of the subject, optionally with a treatment plan. According to some embodiments, the Decision LLM-based agent may be configured to provide explanation for each differential diagnosis generated and for each decision for admission or discharge of the subject, by highlighting the what in the medical history, anamnesis and medical test results led to the generation of the differential diagnosis and decision of admission of discharge.

According to some embodiments the generated summary may be organized at subjective objective assessment plan (SOAP) configuration.

According to some embodiments, recommending treatment plan includes recommending a drug treatment. According to some embodiments the treatment/drug plan may be generated by drug plan LLM-based agent 135.

According to some embodiments, a treatment plan may be recommended during the subject's stay at the emergency department, after the physical examination or after receiving the recommended test and examinations results.

According to some embodiments, the summary including a differential diagnosis, and a decision to discharge with treatment plan or admit with a treatment plan, may be provided after receiving results of the physical examinations or after receiving the test and examinations results.

According to some embodiments, the physical examination may be performed by a human clinician or by an IOT medical device.

According to some embodiments, the additional examinations may include laboratory test/examinations, diagnostic examinations and/or imaging examinations.

According to some embodiments, the system and method may be configured to further determine urgency according to the preliminary measurements, subject's complaint(s), anamnesis and identified relevant medical information after generating comprehensive anamnesis.

According to some embodiments, the computerized system may further include receiving approval of a human clinician of said decision or receiving a different decision regarding the subject's admission or discharge and/or treatment plan.

According to some embodiments, the step of receiving an ID of a subject, extracting medical information of said subject from a medical information system and providing a summarized medical history of the subject, storing said summarized medical history in a database, and receiving results of preliminary examinations obtained from the subject in the emergency department may further include processing the summarized medical history and preliminary examination results to identify medical information relevant to the preliminary examination results.

According to some embodiments, the method may further include reviewing each step by evaluation LLM-based agent 137 and providing a score to each step for enabling future optimization of each of steps 202-218.

According to some embodiments, the method may further include receiving further input from the subject by a human clinician and/or receiving an amended anamnesis by said clinician.

According to some embodiments, the method may further include receiving approval of a human clinician of the recommendations for test and examinations or receiving further or different recommendations for additional examinations from the human clinician.

According to some embodiments, the summarized medical history of the subject may be labeled according to the ID of the subject.

According to some embodiments, the computerized system may include LLM models such as Generative Pretrained Transformer (GPT), sonnet, Claude or any other suitable LLM models.

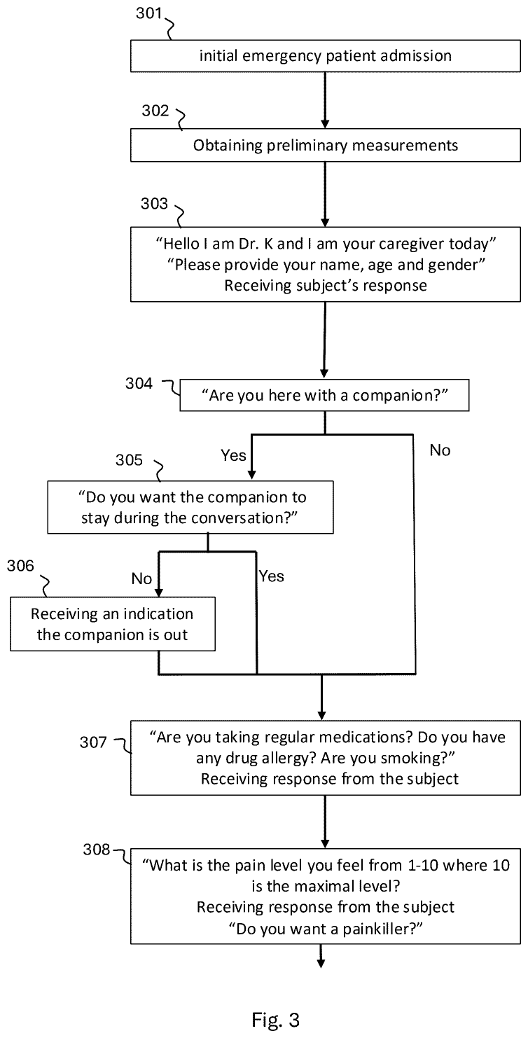

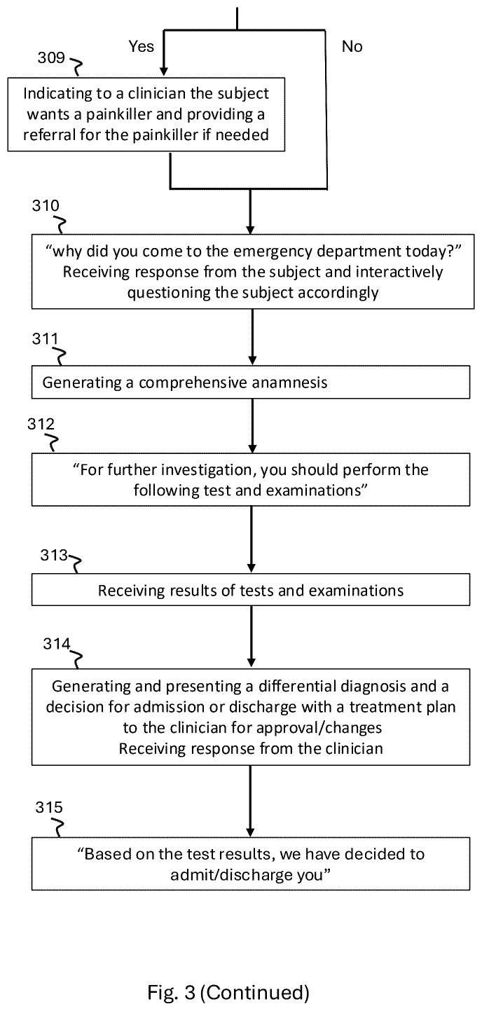

Reference is now made to FIG. 3, schematically illustrating a flowchart describing an example of flow management of a subject, accepted/admitted at an emergency department by a computerized system, according to some embodiments.

As shown in FIG. 3, at step 301, the subject arrives at the emergency department and undergoes an initial emergency patient admission including labeling the main complaint(s) of the patient and uploading the main complaint(s) to the medical information system of the hospital. According to some embodiments, the location of admission may be a room or a closed position including a subject interface device having a display (such as a screen or touch screen), a microphone and a speaker. According to some embodiments, once the subject agreed to receive healthcare by the system, the ID of the subject is fed into the system, to generate the summarized medical history of the patient, as detailed herein. According to some embodiments, at step 302, initial measurements are obtained from the patient by an IoT medical device such as an RFID bracelet, wearable monitoring stickers and pulse oximeter sleeves, or by a human medical staff member. Optionally, the initial measurement may be obtained in parallel by the IoT medical devices as a one click operation and the results may be fed to the medical information system and the database of the system, to generate a processed summarized medical history of the patient by the system. At step 303, a conversation with the system, for example, using a virtual avatar representing the system, starts via the subject interface device, where the virtual avatar may present itself as a virtual clinician and request the patient to provide identifying details, such as name age and gender. At step 304, the virtual avatar may ask if the patient arrived with a companion, for example “Have you came with a companion?”. If the response of the patient is “no”, the virtual avatar proceeds with step 307 and if the patient response is “yes”, at step 306 the virtual avatar asks if the subject wants the companion to stay with him during the conversation with the virtual avatar. In case the subject does not want the companion to stay with him during the conversation, the subject is required to indicate to the virtual avatar the companion is out of the room. The indication may be by writing in the subject interface device or by an audio indication via the microphone. At step 307, the virtual avatar questions the subject regarding the subject's medical history including allergies, drugs and recent surgeries to verify and/or update the preprocessed summarized medical history of the subject. According to some embodiments the questions may also include lifestyle questions such as does the patient is taking drugs, drinking alcohol, smoking, training and is the subject under a specific diet. At step 308, the virtual avatar asks the subject regarding pain. According to some embodiments, the patient is asked if he feels any pain and if the response is yes, the subject is required to rate the level of pain for example from 1-10, 1-5, or on any suitable scale. According to some embodiments, the subject is asked if he wants a painkiller and if the response of the subject is “yes”, at step 309 an indication is sent to a human clinician, and a referral for the painkiller may be provided by the human clinician or by the background LLM-based agent operating the virtual avatar at this step. At step 310, according to some embodiments, the subject may be questioned regarding its main complaint(s), for example “why did you come to the emergency department today?” or the like. At this step, the virtual avatar is operated by the anamnesis LLM-based agent, however the transition between the agents is transparent to the patient. According to some embodiments, this step depends on the response of the patient and the virtual avatar may interactively ask the subject additional questions according to the answers of the subject. According to some embodiments, at step 311, once the virtual avatar finishes to ask the subject regarding its main complaint, the virtual avatar generates, by the Anamnesis LLM-based agent, a comprehensive anamnesis, however the anamnesis may or may not be presented to the subject. According to some embodiments, at step 312, tests and examinations may be presented to the subject. The virtual avatar generates, by the Diagnosis LLM-based agent suggested diagnosis and outputs recommendations by Test recommender LLM-based agent regarding recommended tests and examinations for confirming or ruling-out the possible diagnosis. According to some embodiments, the recommended tests and examinations may be presented to a clinician via the clinician interface device for approval and/or changes. Once the examinations are approved, the virtual avatar presents the required examinations to the subject and the subject is required to complete these test and examinations. According to some embodiment, some of the tests and/or examinations may be taken or conducted in the space/room the in which the system is located. Such examinations may include, for example, blood test, echocardiogram, physical examination, and the like. In this case, the subject is required to wait in the room for a medical staff member to come and conduct the test/examination. In other cases, according to some embodiments, the examinations may take place at a dedicated location, for example, CT, US, MRI, imaging examinations, and the like. In this case the subject is required to go to the required department. According to some embodiments, the referral for each examination may be provided and sent to the dedicated department by the human clinician or possibly by Test recommended LLM-based agent operating the virtual avatar at this step. According to some embodiments, the virtual avatar may provide a short explanation regarding each test or examination required according to the subject wish. In this case, the virtual avatar may ask the subject if he wants a short explanation regarding the examinations and if the subjects answer is “yes”, a short explanation may be presented to the subject regarding each test/examination via the subject interface device. According to some embodiments, at step 313, the results of the tests and examinations are received and at step 314 the virtual avatar may generate a differential prospective/potential diagnosis. According to some embodiments, the differential diagnosis is first presented to the human clinician for approval or changes. According to some embodiments, at this step, additional examinations may be required. In this case the additional examinations required are presented to the subject. In case no further examination is required, a decision regarding admission of the subject (with or without a treatment plan) or discharge of the subject (with or without a treatment plan) is generated. According to some embodiments, the decision is presented first to the human clinician for approval or changes via the clinician interface device and once approved, at step 315 the differential diagnosis and the decision with the treatment plan are presented to the subject via the subject interface device. According to some embodiments the virtual avatar may generate an admission or discharge letter for the clinician to sign up and to provide to the subject.

According to some embodiments, during the subject stay at the emergency department a visual stages bar may be presented to the subject via the subject interface device providing an indication to the subject regarding the stage he is in with regard to the emergency department treatment stages. For example, the visual stages bar may indicate the subject he is in the medical history questioning stage, main complaint questioning stage, test and examination recommendation stage, waiting for test and examinations results stage, treatment plant stage, decision stage and the like.

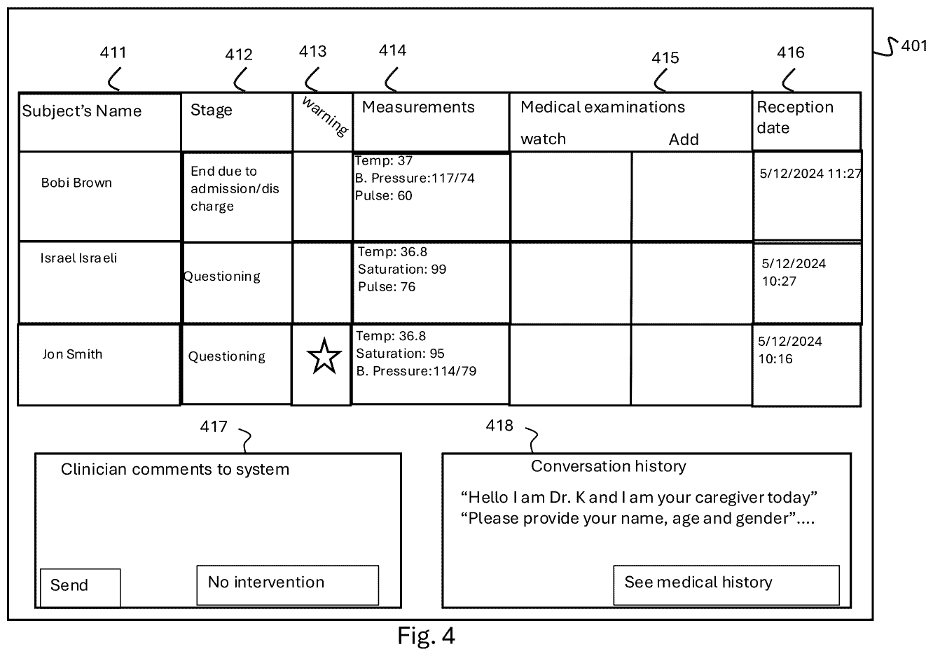

FIG. 4 schematically shows an example of a clinician interface device screen of a computerized system as presented to a clinician in the ED, according to some embodiments. As can be seen, screen 401 is a clinician interface device screen presenting to a clinician the subjects treated by the computerized system in the ED. According to some embodiments, the subjects names are presented in subject's name field 411, in this example a list of three subjects named “Bobi Brown”, “Israel Israeli” and “Jon Smith” is presented. The stage the subject is in, is presented in field 412. For example, Bobi Brown is at the end stage due to receiving a decision regarding admission/discharge. Warning and/or notifications to the clinician are presented in warning field 413 for indicating a warning requiring the clinician attention, for example, for requesting an immediate intervention, painkiller indication etc., at this example it can be seen that a warning is shown for subject “Jon Smith”. The preliminary measurements obtained from the subject are presented in measurements field 414, for example, Bobi Brown measurements show body temperature of 37 degrees, Blood pressure of 117/74, and heart rate of 60 pulses per minute. A medical examination field 415 present the option of watching the results of available medical examinations and provide an option to the clinician to add additional medical examination required. The date and hour of reception of the subject is presented at field reception date 416. According to some embodiments, a clinician comments to system field 417 is presented where the clinician may send changes approvals or any comments to the system. According to some embodiments, the conversation history with the subject is presented to the clinician at field conversation history 418. In this field the clinician may see the summarized medical history of the subject by pressing the “see medical history” key.

According to some embodiments, there are provided an AI-powered Emergency Department patient flow management system and method. In some embodiments, the disclosed system and methods can free physicians from extensive medical reading and documentation, thus maximizing patient-doctor interaction time. In some embodiments, the system is based on a plurality of LLM agents, trained and optimized to summarize patient data, conduct comprehensive anamnesis, triage patients by identifying urgency, recommend necessary examinations such as blood tests and imaging, make advanced treatment recommendations and informed decisions about patient admission or discharge.

According to some embodiments, upon arriving at the Emergency Department (ED), patients are triaged, for example, by a nurse and, then may be guided to the Digital ED section, including the computerized flow management system. The identification of the patient is verified (for example, by RFID tag attached to their wrist), and a structured conversation with the AI LLM agent(s) of system is facilitated, designed to obtain a detailed understanding of the subject's current condition. Simultaneously, vital signs and physical examination may be carried out. Through real-time integration with the Electronic Medical Record (EMR), the AI system may retrieve and collate relevant past medical history and community care records, supplementing the information obtained during its conversation with the patient. This comprehensive understanding of the patient's state allows the system to propose an initial course of further workup, such as imaging, blood tests, and other diagnostic measures, as detailed herein.

According to some embodiments, there is provided an AI based computerized system for full patient flow management through an emergency department process, the system includes:
a plurality of large language models (LLMs) including:
at least one LLM configured to upon receiving an ID of a subject, extract medical information of said subject from a medical information system and provide a summarized medical history of the subject, storing said summarized medical history in a database;
at least one LLM configured to receive results of preliminary physical examinations obtained from the subject in the emergency department;
at least one LLM configured to preprocess the summarized medical history and the preliminary physical examination results to identify medical information relevant to the preliminary examinations results;
at least one LLM configured to receive input from the subject, by interactively questioning the subject regarding subject's complaint thereby generating comprehensive anamnesis according to subject's complaint and the preprocessed summarized medical history; and
at least one LLM configured to receive results of a physical examination;
at least one LLM configured to output recommendations regarding additional examinations;
at least one LLM configured to receive the additional examinations results;
at least one LLM configured to provide a summary comprising:
   a. a differential diagnosis; and
   b. a decision regarding admission of the subject to one of a hospital department with a treatment plan or discharge of the subject with a treatment plan.

According to some embodiments, at least one LLM is configured to provide a summary comprising a differential diagnosis, and a decision to discharge with treatment plan or admit with a treatment plan, is configured to provide said summary after the results of the physical examinations are received or after the results of the additional examinations are received.

According to some embodiments, the physical examination may be performed by a human clinician or by an IOT medical device.

According to some embodiments, the plurality of LLMs further include at least one LLM configured to recommend a treatment plan during the subject's stay at the emergency department, after the physical examination or after the additional examination results are received.

According to some embodiments, the at least one LLM is configured to provide a summary comprising a differential diagnosis and a decision is configured to receive approval of a human clinician of said decision or to receive a different decision regarding the admission or discharge of the subject and treatment plan.

According to some embodiments, the additional examinations may include laboratory examinations and/or imaging examinations.

According to some embodiments, the treatment plan may include a drug treatment.

According to some embodiments, the plurality of LLMs may be trained by prompt engineering.

According to some embodiments, the plurality of LLMs may further include at least one LLM configured to determine urgency according to the subject's complaint, anamnesis and identified relevant medical information.

According to some embodiments, the at least one LLM may be configured to extract medical information of said subject from a medical information system and provide a summarized medical history of the subject, storing said summarized medical history in a database is further configured to receive results of preliminary examinations obtained from the subject in the emergency department.

According to some embodiments, the at least one LLM may be configured to receive input from the subject, by interactively questioning the subject regarding subject's complaint is configured to receive a human clinician input regarding the input from the subject and/or amending the provided anamnesis.

According to some embodiments, the at least one LLM may be configured to output recommendations regarding additional examinations is configured to receive approval of a human clinician of the recommendations for additional examinations or receive further or different recommendations for additional examinations from the human clinician.

According to some embodiments, the plurality of LLMs may further include at least one LLM configured to review each LLM operation and output and provide a score to each LLM for said operation and output enabling future optimization of each LLM operation and results.

According to some embodiments, the summarized medical background of the subject may be labeled according to the ID of the subject.

According to some embodiments, the AI based computerized system may include Generative Pretrained Transformer (GPT).

According to some embodiments, there is provided an AI-based computerized method for full patient flow management through an emergency department process, the method includes:
upon receiving an ID of a subject, extracting medical information of said subject from a medical information system and providing a summarized medical history of the subject, storing said summarized medical history in a database, and receiving results of preliminary examinations obtained from the subject in the emergency department;
preprocessing the summarized medical history and preliminary examination results to identify medical information relevant to the preliminary examination results;
receiving input from the subject by interactively questioning the subject regarding subject's complaint thereby generating comprehensive anamnesis according to subject's complaint and the preprocessed summarized medical history;
receiving results of a physical examination; and
outputting recommendations regarding additional examinations;
receiving the additional examinations results;
providing a summary including:
   a. a differential diagnosis; and
   b. a decision regarding admission of the subject with a treatment plan or discharge of the subject with a treatment plan.

According to some embodiments, the method may further include receiving approval of a human clinician of said decision or receiving a different decision regarding the subject's admission or discharge and treatment plan.

According to some embodiments, recommending treatment plan includes recommending a drug treatment.

According to some embodiments, the method may further include recommending a treatment plan during the subject's stay at the emergency department, after the physical examination or after receiving the additional examination results.

According to some embodiments, providing a summary may include a differential diagnosis, and a decision to discharge with treatment plan or admit with a treatment plan, is done after receiving results of the physical examinations or after receiving the additional examinations results.

According to some embodiments, the method may further include determining urgency according to the subject's complaint, anamnesis and/or identified relevant medical information after generating comprehensive anamnesis.

According to some embodiments, the method may further include reviewing one of or each of the steps and providing a score to each step for enabling future optimization of each step.

According to some embodiments, the method may further include receiving further input from the subject by a human clinician and/or receiving an amended anamnesis by said clinician.

According to some embodiments, the method may further include receiving approval of a human clinician of the recommendations for additional examinations or receiving further or different recommendations for additional examinations from the human clinician.

According to some embodiments, the method may further include labeling the summarized medical background of the subject according to the ID of the subject.

According to some embodiments, the computerized models disclosed herein utilize Machine learning (ML) and Artificial intelligence (AI) tools, including any type of suitable algorithms, such as, for example, but not limited to: transformers, artificial neural network(s) (ANN), such as convolutional neural network (CNN), recurrent neural network (RNN), long-short term memory (LSTM), auto-encoder (AE), generative adversarial network (GAN), Retrieval Augmented Generation (RAG), Reinforcement-Learning (RL), support vector machine (SVM), decision tree (DT), random forest (RF), and the like. Both “supervised” and “unsupervised” methods may be implemented.

According to some embodiments, there is provided a non-transitory memory device, wherein modules of instruction code are stored, and at least one processor associated with the memory device, and configured to execute the modules of instruction code, whereupon execution of said modules of instruction code, the at least one processor is configured to execute the patient flow management methods disclosed herein.

According to some embodiments, there is provided a non-transitory computer-readable storage medium. The storage medium stores instructions that cause one or more processors to implement one more of the AI agents specified methods for patient flow management in emergency department.

### Enumerated Items

1. A computerized system for full patient flow management through an emergency department process, the system comprising one or more processing units executing code instructions configured to:
   receive results of preliminary measurements obtained from a subject in the emergency department;
   interactively provide queries to the subject and generate a comprehensive anamnesis utilizing:
   a background LLM-based agent, configured to provide queries related to the medical history of the subject and receive medical history related input from the subject; and
   an anamnesis LLM-based agent configured to provide queries related to complaint(s) of the subject, and receive complaint related input from the subject;
      wherein the comprehensive anamnesis is generated based on the complaint related input from the subject and the preliminary measurements;
   generate potential diagnosis utilizing a diagnosis LLM-based agent, based on the comprehensive anamnesis;
   output recommendations regarding one or more medical examinations, utilizing a test recommender LLM-based agent, said agent is configured to receive the generated potential diagnosis and output recommended medical examinations for confirming or ruling-out said potential diagnosis;
   receive results of one or more of the recommended medical examinations; and
   generate a summary utilizing a decision LLM-based agent based on the recommended medical examinations results and the comprehensive anamnesis, wherein the summary comprises:
      a. a differential diagnosis; and/or
      b. a recommendation regarding admission or discharge of the subject from the emergency department.
2. The system of item 1, wherein the one or more processing units are further configured to execute code instructions configured to:
   upon receiving an ID of the subject, extract medical information of said subject from a medical information system; and
   generate and store, on a database, a summarized medical history of the subject, by a pre-summary LLM-based agent comprising a plurality of collecting agents, said collecting agents comprise one or more LLM-based agents and/or one or more extracting agents, each collecting agent is configured to extract and summarize information related to a defined subject-related parameter, said pre-summary LLM-based agent generates the summarized medical history based on the summaries provided by the collecting agents.
3. The system of item 1 or 2, wherein the one or more processing units are further configured to process the summarized medical history and/or preliminary measurements results to identify medical information relevant to the preliminary measurements results.
4. The system of item 2, wherein the background LLM-based agent is further configured to verify and/or update the information in the processed summarized medical history, based on the medical history related input.
5. The system of any one of items 2-4, wherein the comprehensive anamnesis is further based on the verified and/or updated processed summarized medical history.
6. The system of any one of items 2-5, wherein the potential diagnosis is further based on the verified and/or updated processed summarized medical history.
7. The system of any one of items 2-6, wherein the decision is further based on the verified and/or updated summarized medical history.
8. The system of any one of items 2-7, wherein the defined medical parameters comprises chronic conditions, past surgical history, changes and discontinuations of medications, allergies, medical history, acute medications, ambiguous medications, timeline medical history, review of biological systems, imaging and diagnostic results.
9. The system any one of items 2-8, wherein the summarized medical history is generated in about 5-10 minutes.
10. The system of any one of items 1-9, wherein the summary further comprises:
   instructions to repeat execution of the anamnesis LLM-based agent, the diagnosis LLM-based agent, and/or the test recommender LLM-based agent; and/or
   instructions to add or repeat recommended medical examinations.
11. The system of any one of items 1-10, wherein the one or more processing units are further configured to execute code instructions configured to receive results of a physical examination, after generating the comprehensive anamnesis.
12. The system of item 11, wherein the physical examination is done by a human clinician or by an IOT medical device.
13. The system of any one of items 1-12, wherein the recommended medical examinations comprise lab tests, imaging, and/or physical examinations.
14. The system of item 13, wherein the one or more processing units further executes code instructions configured to analyze the imaging examinations.
15. The system of any one of items 1-14, wherein the one or more processing units execute code instructions configured to present the recommended medical examinations and summary via an interface module, to the subject and/or to a health care provider.
16. The system of any one of items 1-15, wherein the preliminary measurements comprises body temperature, heart rate, oxygen saturation, diastolic blood pressure, systolic blood pressure, sugar level, pain level and weight.
17. The system of any one of items 1-16, wherein the preliminary measurements are conducted automatically or semi-automatically by an IOT medical device or by a health care provider.
18. The system of any one of items 1-17, wherein the one or more processing units execute code instructions configured to recommend a treatment plan during the subject's stay at the emergency department, or after the one or more medical examinations results are received, utilizing a drug plan LLM-based agent.
19. The system of item 18, wherein the treatment plan comprises a drug treatment.
20. The system of any one of items 1-19, wherein the decision LLM-based agent is configured to receive an approval or change from a human clinician to the recommendation regarding the admission or discharge of the subject.
21. The system of any one of items 1-20, wherein the anamnesis LLM-based agent is configured to receive a human clinician approval or changes regarding the complaint related input from the subject and/or amending the provided anamnesis.
22. The system of any one of items 1-21, wherein the test recommender LLM-based agent is configured to receive approval or changes or additions from a human clinician regarding the one or more medical examinations recommended.
23. The system of any one of items 1-22, wherein the one or more processing units execute code instructions configured to review one or more of the LLM-based agents operation and output and provide a score to the one or more LLM-based agents for said operation and output, utilizing an evaluation LLM-based agent.
24. The system of item 23, wherein the evaluation LLM-based agent is configured to evaluate the output of one or more LLM-based agents with respect to content structure and linguistic integration using guardrails implemented to ensure that the LLM-based agent operates safely, ethically and in accordance with the LLM-based agent's defined objectives.
25. The system any one of items 1-24, wherein the LLM in the computerized system is Generative Pretrained Transformer (GPT).
26. A computerized method for full patient flow management through an emergency department process, the method comprising:
   receiving results of preliminary measurements obtained from the subject in the emergency department;
   interactively providing queries to the subject and generating a comprehensive anamnesis utilizing:
   a background LLM-based agent, configured to provide queries related to the medical history of the subject and receive medical history related input from the subject; and
   an anamnesis LLM-based agent configured to provide queries related to complaint(s) of the subject, and receive complaint related input from the subject;
      wherein the comprehensive anamnesis is generated based on the complaint related input from the subject and the preliminary measurements;
   generating potential diagnosis utilizing a diagnosis LLM-based agent, based on the comprehensive anamnesis;
   outputting recommendations regarding one or more medical examinations, utilizing a test recommender LLM-based agent, said agent is configured to receive the generated potential diagnosis and output recommended medical examinations for confirming or ruling-out said potential diagnosis;
   receiving results of one or more of the recommended medical examinations; and
   generating a summary utilizing a decision LLM-based agent based on the recommended medical examinations results and the comprehensive anamnesis, wherein the summary comprises:
      a. a differential diagnosis; and/or
      b. a recommendation regarding admission or discharge of the subject from the emergency department.
27. The computerized method of item 26, further comprising:
   upon receiving an ID of a subject, extracting medical information of said subject from a medical information system;
   generating and storing, on a database, a summarized medical history of the subject, by a pre-summary LLM-based agent comprising a plurality of collecting agents, said collecting agents comprise one or more LLM-based agents and/or one or more extracting agents, each collecting agent is configured to extract and summarize information related to a defined subject-related parameter, said pre-summary LLM-based agent generates the summarized medical history based on the summaries provided by the collecting agents.
28. The computerized method of item 26 or 27, further comprising processing the summarized medical history and/or preliminary measurements results by the pre-summary LLM-based agent to identify medical information relevant to the preliminary measurements results.
29. The computerized method of any one of items 26-28, wherein the background LLM-based agent is further configured to verify and/or update the information in the processed summarized medical history, based on the medical history related input.
30. The computerized method of any one of items 26-29, wherein the comprehensive anamnesis is further based on the verified and/or updated processed summarized medical history.
31. The computerized method of any one of items 26-30, wherein the potential diagnosis is further based on the verified and/or updated processed summarized medical history.
32. The computerized method of any one of items 26-31, wherein the summary is further based on the verified and/or updated summarized medical history.
33. The computerized method of any one of items 26-32, wherein the summary further comprises:
   instructions to repeat execution of the background LLM-based agent, the anamnesis LLM-based agent, the diagnosis LLM-based agent, and/or the test recommender LLM-based agent; and/or
   instructions to add or repeat recommended medical examinations.
34. The computerized method of item any one of items 26-33, wherein the summary is generated after receiving results of the one or more medical examinations or the summary is regenerated after receiving the results of the added or repeated recommended medical examinations.
35. The computerized method of any one of items 26-34, further comprising receiving results of a physical examination, after generating the comprehensive anamnesis.
36. The computerized method of claim 35, wherein the physical examination is done by a human clinician or by an IOT medical device.
37. The computerized method any one of items 26-36, further comprising receiving an approval or change from a human clinician to the recommendation regarding the admission or discharge of the subject.
38. The computerized method any one of items 26-37, further comprising recommending a treatment plan.
39. The computerized method any one of items 26-38, wherein the treatment plan comprises a drug treatment.
40. The computerized method of any one of items 26-39, wherein the treatment plan is recommended during the subject's stay at the emergency department, or after receiving the one or more medical examination results.
41. The computerized method of any one of items 26-40, wherein the one or more medical examinations comprising lab tests imaging and/or physical examinations.
42. The computerized method of any one of items 26-41, further comprising determining urgency based on the subject's complaint, anamnesis and identified medical information relevant to the preliminary measurements results, after generating the comprehensive anamnesis.
43. The computerized method of any one of items 26-42, further comprising reviewing one or more of the LLM-based agents operation and output and providing a score to the one or more LLM-based agents for said operation and output, utilizing an evaluation LLM-based agent.
44. The computerized method of item 43, wherein reviewing one or more LLM-based agents operation and output comprises evaluating the output of the respective LLM-based agent with respect to content structure and linguistic integration using guardrails implemented to ensure that the LLM-based agent operates safely, ethically and in accordance with the LLM-based agent's defined objectives.
45. The computerized method of any one of items 26-44, further comprising receiving further complaint input from the subject by a human clinician and/or receiving an amended anamnesis by said clinician.
46. The computerized method of any one of items 26-45, further comprising receiving approval or changes or additions from a human clinician regarding the one or more medical examinations recommended.
47. A non-transitory computer-readable medium having stored thereon instructions that cause a processor to perform the method of any one of items 26-46.

Unless otherwise defined the various embodiment of the present invention may be provided to an end user in a plurality of formats, platforms, and may be outputted to at least one of a computer readable memory, a computer display device, a printout, a computer on a network, a tablet or a smartphone application or a user. Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software (or program code), selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “processor” “hardware processor” or “computer” on a “computer network”, it should be noted that optionally any device featuring a data processor and/or the ability to execute one or more instructions may be described as a computer, including but not limited to a PC (personal computer), a server, a minicomputer, a cellular telephone, a smart phone, a PDA (personal data assistant), a pager. Any two or more of such devices in communication with each other, and/or any computer in communication with any other computer, may optionally comprise a “computer network”.

Embodiments of the present invention may include apparatuses for performing the operations herein. This apparatus may be specially constructed for the desired purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The invention may be described in the general context of computer-executable instructions, such as program modules, being executed by a computer. Generally, program modules include routines, programs, objects, components, data structures, and so forth, which perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1—Assessment of Patient Flow Management in Emergency Department, Using LLM-Based Computerized System

An observational study was carried out at the adult emergency department over three months. The experiment focused on patients suitable for non-urgent care to minimize any potential risks from LLM involvement. The LLM model used was ChatGPT-4, trained on a large dataset without specific task objectives.

During the experiment, physicians provided standard care while an independent assessor simulated case discussions with the LLM model, inputting patients' medical histories, examination findings, test results and more. Assessments were generated by the model, generating differential diagnoses, suggested follow-up, and disposition plans, which were compared to physicians' documentation. Concordance rates were quantified for examination types recommended, final/differential diagnoses, and disposition decisions.

A total of 33 patients were included in the study.

Patients were eligible if they met the following criteria: 18 years or older; able to communicate in either Hebrew or English; and assessed at triage with an Emergency Severity Index (ESI) score of 3 or higher, indicating their condition required evaluation or monitoring but no immediate medical intervention.

Patients were excluded if they: required urgent or emergent medical care; had severe communication impairments or mental health disorders that precluded informed consent; or were pregnant. A total of 33 eligible patients were consecutively enrolled during the study period. All participants provided written informed consent after receiving a detailed explanation of study procedures from the research team. The mean age was 34.96 years with a standard deviation of 12.63 years. Most of the patients (60.6%, n=20) were male.

Table 1 below summarize the descriptive statistics of the experiment setting, including, Sociodemographic data, clinical data, main complaint(s) and medical histories of the participants.

TABLE 1

| | | |
|---|---|---|
| Variable | Mean/N | SD/% |

Sociodemographic data

| | | |
|---|---|---|
| Age (mean (SD)) | 34.96 | 12.63 |
| Gender (male) (n (%)) | 20 | 60.6 |

Clinical data
Vital signs in admission

| | | |
|---|---|---|
| Systolic BP, Mean (Range) | 123.68 (97-167) | 16.36 |
| Diastolic BP, Mean (Range) | 79.37 (56-107) | 11.01 |
| Fever, Mean (Range) | 36.84 (36-38) | 0.44 |
| Pulse | 83.91 (53-130) | 19.57 |
| SaO2 | 97.9 (94-100) | 1.46 |

Main complaint

| | | |
|---|---|---|
| Chest pain | 6 | 18.18 |
| Limbs/Neck pain | 4 | 12.12 |
| Headache | 3 | 9.09 |
| Upper respiratory symptoms | 6 | 18.18 |
| Abdominal pain/GI symptoms | 6 | 18.18 |
| Neck/Back pain | 2 | 6.06 |
| Other | 6 | 18.18 |

Medical history

| | | |
|---|---|---|
| None | 21 | 63.64 |
| Herniated disk | 2 | 6.06 |
| Arthritis/Gout | 2 | 6.06 |
| Mental illness | 2 | 6.06 |
| ADHD | 2 | 6.06 |
| Crohn's disease | 2 | 6.06 |
| Other | 2 | 6.06 |

For each case, demographic information was collected, including age, sex, primary language, and reason for ED visit. Vital signs and clinical notes from the physician's initial assessment were also recorded. This comprehensive dataset allowed for thorough evaluation of the system's performance and safety across a range of non-urgent ED presentations. Strict protocols were followed to protect patient privacy and data security throughout the study.

All statistical analyses were performed using SPSS software version 29.0. Continuous variables were reported as means and standard deviations or medians and interquartile ranges based on distribution. Categorical variables were reported as frequencies and percentages.

To evaluate the concordance between the system's recommendations and physicians' assessments, percentages of agreement were calculated for examination recommendations, final and differential diagnoses, and disposition decisions. Confidence intervals of 95% were determined using the Wilson score method without continuity correction for binomial proportions.

Independent sample t-tests were used to compare quantitative variables (e.g. age) between groups where system and physician decisions aligned versus did not align. Chi-squared tests and Fisher's exact tests were used for categorical variables (e.g. gender) as appropriate based on expected cell counts. One-way ANOVA with Bonferroni post-hoc correction was used to compare diagnostic test recommendation accuracy across age categories. Upon admission to the ED, each patient received standard clinical care per hospital protocols. In addition, an independent secondary physician conducted a simulated evaluation using the system's model.

The secondary physician inputted each patient's demographic information, medical history, and current health details into a natural language conversation with the LLM agent. This included details captured from the physical exam and any diagnostic tests performed as part of the standard ED workup. The system then “simulated” the clinical decision-making process by generating initial medical assessments, suggesting additional exams or tests, integrating results, and offering medical summaries, differential diagnoses, and treatment recommendations via the conversational interface.

### Results

The LLM agent results matched physicians' examination selections in 73% of instances. It correctly identified the final diagnosis in 91% of cases and top differential in 79%. Disposition decisions aligned in 61% of encounters. Physicians intervened most often on the history-taking (12 cases) and test recommendations (13 cases). No significant differences were found between accuracy and demographic factors.

The average systolic blood pressure on admission was 123.68 mmHg with a range from 97-167 mmHg. The mean diastolic blood pressure was 79.37 mmHg with a range of 56-107 mmHg. The average fever reading was 36.84° C. ranging from 36-38° C. The average pulse was 83.91 beats/minute ranging from 53-130 beats/minute. The average oxygen saturation was 97.9% ranging from 94-100%. Regarding the main complaints, the most common were chest pain (18.18%, n=6), upper respiratory symptoms (18.18%, n=6), abdominal pain/GI symptoms (18.18%, n=6), and limbs/neck pain (12.12%, n=4). Most patients (63.64%, n=21) did not have any significant past medical history.

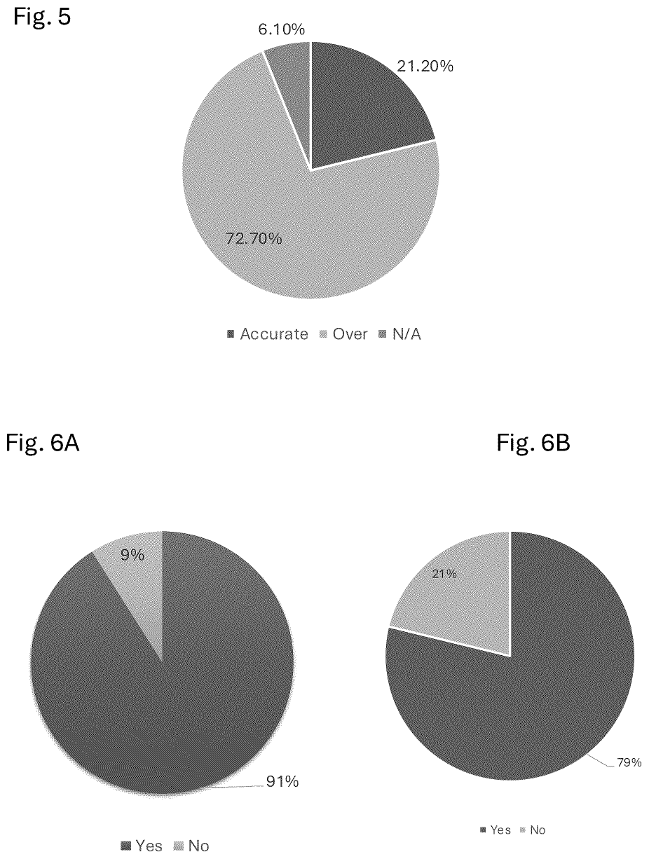

FIG. 5 schematically shows a comparison between results a of medical examination recommendations made by the LLM agent and physicians. As can be seen, a significant majority of the LLM recommendations (72.70%), were in alignment with those made by physicians, indicating a high level of accuracy in the LLM agent recommendations. However, there was 21.20%, where the LLM recommended more examinations than the physicians did.

The most common over-recommended examinations were chest x-rays, which were excessive in 4 cases. Orthopedic imaging such as x-rays were also over-suggested in 4 cases. Neck/head CT scans were excessively recommended in 3 cases. Abdominal ultrasound was over-recommended in 5 cases. Urine culture tests were over-recommended in 3 cases, and coronavirus/respiratory virus antigen or nasal swab tests in 6 cases.

FIG. 6A schematically shows the rate of matching between the recommended differential diagnosis of the LLM and the physician diagnosis in the results of the experiment of example 1, according to some embodiments. A significant majority of the cases studied, precisely 91%, demonstrated a positive match.

FIG. 6B schematically shows the rate of matching priority differential diagnoses between the LLM and physicians. The results showed that a significant majority, 79%, of the priority differential diagnoses made by the LLM were in agreement with those made by physicians.

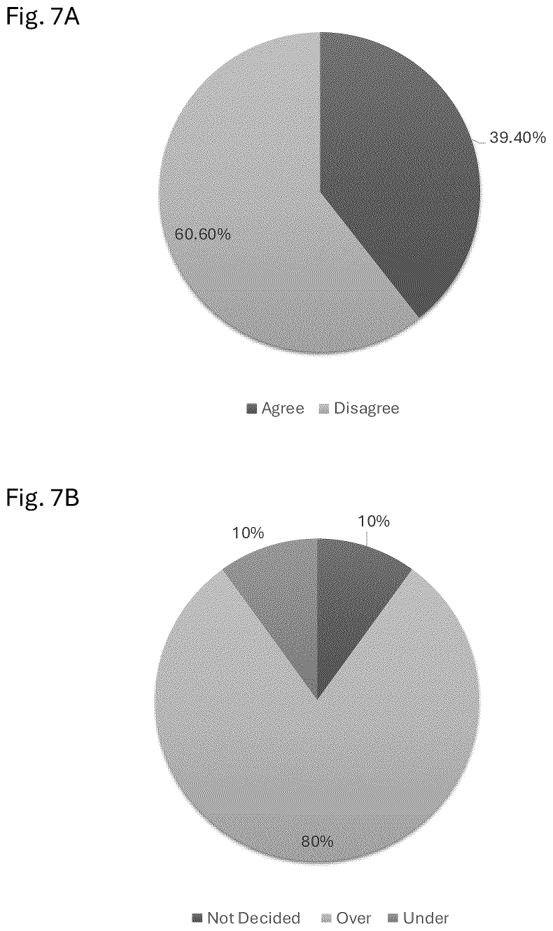

FIG. 7A schematically shows the rate of agreement on patient discharge decisions between the LLM and physicians. It can be seen that in 60.60% of cases, LLM disposition recommendation aligned with the decision ultimately made by physicians.

FIG. 7B schematically shows the discrepancies in patient discharge decisions where the LLM and physicians disagreed (n=20). As can be seen in FIG. 7B, the majority (80%) of discrepant decisions represented instances where the LLM overestimated care needs by recommending hospitalization for patients that the treating clinicians determined could be safely discharged. The remaining 20% was split as follows: 10% comprised cases where the LLM may have underestimated needs by suggesting discharge when physicians elected to admit, and the other 10% represented cases where the LLM did not provide a definitive discharge recommendation.

The physician interventions required to adjust the LLM's decisions were evaluated. In 12 cases, the physician needed to correct or amend the patient's medical history that was provided to the LLM model. Additionally, in 3 cases the physician found it necessary to modify the history obtained through the LLM question-asking. The physical examination recommendation generated by the LLM also required physician input, with adjustments made in 3 cases. Most commonly, the panel of diagnostic tests suggested by the LLM warranted refinement by the treating clinician, with changes implemented in 13 cases.

No significant association was found between patient gender and accuracy of disposition decision (hospitalization vs discharge) (p=0.403). The results were also examined categorically for this variable but no significant difference was detected (p=0.521). Similarly, no relationship was seen between patient age and accuracy of discharge decision (p=0.280), and categorical examination of this variable also showed no variation (p=0.513).

Patient gender was also not significantly related to accuracy of differential diagnosis (p=0.876). No strong association was found for the variable defining differential diagnosis matched to the physician as top priority either (p=0.815). Regarding age, no link was detected between this factor and differential diagnosis precision (p=0.272), nor when defining the variable as matching the physician's top differential (p=0.117).

No meaningful relationship was seen between decision accuracy and patient's vital signs upon admission. However, diagnostic test recommendations differed by age-older patients (Mean=46.6, SD=6.8) showed higher accuracy from the model compared to younger patients (Mean=33.2, SD=12.1) who received more excessive suggestions (p=0.001).

Conclusions: In this prospective evaluation, using extensive real patient data, the LLM system demonstrated strong concordance with expert clinical assessments for key emergency medicine judgments.

1.-47. (canceled)

48. A computerized method for full patient flow management through an emergency department process, the method comprising:
receiving results of preliminary measurements obtained from a subject patient in the emergency department;
interactively providing queries to the subject and generating a comprehensive anamnesis utilizing:
a background LLM-based agent, configured to provide queries related to the medical history of the subject and receive medical history related input from the subject; and
an anamnesis LLM-based agent configured to provide queries related to complaint(s) of the subject, and receive complaint related input from the subject;
wherein the comprehensive anamnesis is generated based on the complaint related input from the subject and the preliminary measurements;
generating potential diagnosis utilizing a diagnosis LLM-based agent, based on the comprehensive anamnesis;
outputting recommendations regarding one or more medical examinations, utilizing a test recommender LLM-based agent, said agent is configured to receive the generated potential diagnosis and output recommended medical examinations for confirming or ruling-out said potential diagnosis;
receiving results of one or more of the recommended medical examinations; and
generating a summary utilizing a decision LLM-based agent based on the recommended medical examinations results and the comprehensive anamnesis, wherein the summary comprises:
a. a differential diagnosis; and/or
b. a recommendation regarding admission or discharge of the subject from the emergency department.

49. The computerized method of claim 48, further comprising:
upon receiving an ID of a subject, extracting medical information of said subject from a medical information system;
generating and storing, on a database, a summarized medical history of the subject, by a pre-summary LLM-based agent comprising a plurality of collecting agents, said collecting agents comprise one or more LLM-based agents and/or one or more extracting agents, each collecting agent is configured to extract and summarize information related to a defined subject-related parameter, said pre-summary LLM-based agent generates the summarized medical history based on the summaries provided by the collecting agents.

50. The computerized method of claim 49, further comprising processing the summarized medical history and preliminary measurements results by the pre-summary LLM-based agent to identify medical information relevant to the preliminary measurements results.

51. The computerized method of claim 49, wherein the background LLM-based agent is further configured to verify and/or update the information in the processed summarized medical history, based on the medical history related input.

52. The computerized method of claim 51, wherein one or more of: the comprehensive anamnesis, the potential diagnosis, the decision are further based on the verified and/or updated processed summarized medical history.

53. The computerized method of claim 49, wherein the defined subject related parameters comprise chronic conditions, past surgical history, changes and discontinuations of medications, allergies, medical history, acute medications, ambiguous medications, timeline medical history, review of biological systems, imaging and diagnostic results, or any combinations thereof.

54. The computerized method of claim 48, wherein the summary further comprises:
instructions to repeat execution of the background LLM-based agent, the anamnesis LLM-based agent, the diagnosis LLM-based agent, and/or the test recommender LLM-based agent; and/or
instructions to add or repeat recommended medical examinations.

55. The computerized method of claim 54, wherein the summary is generated after receiving results of the one or more medical examinations or the summary is regenerated after receiving the results of the added or repeated recommended medical examinations.

56. The computerized method of claim 48, further comprising receiving results of a physical examination, after generating the comprehensive anamnesis, wherein said physical examination is performed by a human clinician or by an IOT medical device.

57. The computerized method of claim 48, further comprising receiving an approval or change from a human clinician for the recommendation regarding the admission or discharge of the subject.

58. The computerized method of claim 48, further comprising recommending a treatment plan.

59. The computerized method of claim 58, wherein the treatment plan is recommended during the subject's stay at the emergency department, or after receiving the one or more medical examination results.

60. The computerized method of claim 48, wherein the one or more medical examinations comprises lab tests, imaging and/or physical examinations.

61. The computerized method of claim 48, further comprising determining urgency based on the subject's complaint, anamnesis and identified medical information relevant to the preliminary measurements results, after generating the comprehensive anamnesis.

62. The computerized method of claim 48, further comprising presenting the recommended medical examinations and summary to the subject and/or to a health care provider.

63. The computerized method of claim 48, further comprising receiving further complaint input from the subject by a human clinician and/or receiving an amended anamnesis by said clinician.

64. The computerized method of claim 48, further comprising receiving approval, changes and/or additions from a human clinician regarding the one or more recommended medical examinations.

65. The computerized method of claim 48, further comprising reviewing one or more of the LLM-based agents operation and output and providing a score to the one or more LLM-based agents for said operation and output, utilizing an evaluation LLM-based agent.

66. The computerized method of claim 65, wherein reviewing one or more LLM-based agents operation and output comprises evaluating the output of the respective LLM-based agent with respect to content structure and linguistic integration using guardrails implemented to ensure that the LLM-based agent operates safely, ethically and in accordance with the LLM-based agent's defined objectives.

67. A computerized system for full patient flow management through an emergency department process, the system comprising one or more processing units executing code instructions configured to:
receive results of preliminary measurements obtained from a subject patient in the emergency department;
interactively provide queries to the subject and generate a comprehensive anamnesis utilizing:
a background LLM-based agent, configured to provide queries related to the medical history of the subject and receive medical history related input from the subject; and
an anamnesis LLM-based agent configured to provide queries related to complaint(s) of the subject, and receive complaint related input from the subject;
wherein the comprehensive anamnesis is generated based on the complaint related input from the subject and the preliminary measurements;
generate potential diagnosis utilizing a diagnosis LLM-based agent, based on the comprehensive anamnesis;
output recommendations regarding one or more medical examinations, utilizing a test recommender LLM-based agent, said agent is configured to receive the generated potential diagnosis and output recommended medical examinations for confirming or ruling-out said potential diagnosis;
receive results of one or more of the recommended medical examinations; and
generate a summary utilizing a decision LLM-based agent based on the recommended medical examinations results and the comprehensive anamnesis, wherein the summary comprises:
a. a differential diagnosis; and/or
b. a recommendation regarding admission or discharge of the subject from the emergency department.

68. A non-transitory computer-readable medium having stored thereon instructions that cause a processor to:
receive results of preliminary measurements obtained from a subject in the emergency department;
interactively provide queries to the subject and generate a comprehensive anamnesis utilizing:
a background LLM-based agent, configured to provide queries related to the medical history of the subject and receive medical history related input from the subject; and
an anamnesis LLM-based agent configured to provide queries related to complaint(s) of the subject, and receive complaint related input from the subject;
wherein the comprehensive anamnesis is generated based on the complaint related input from the subject and the preliminary measurements;
generate potential diagnosis utilizing a diagnosis LLM-based agent, based on the comprehensive anamnesis;
output recommendations regarding one or more medical examinations, utilizing a test recommender LLM-based agent, said agent is configured to receive the generated potential diagnosis and output recommended medical examinations for confirming or ruling-out said potential diagnosis;
receive results of one or more of the recommended medical examinations; and
generate a summary utilizing a decision LLM-based agent based on the recommended medical examinations results and the comprehensive anamnesis, wherein the summary comprises:
a. a differential diagnosis; and/or
b. a recommendation regarding admission or discharge of the subject from the emergency department.
